(19) United States
(12) Patent Application Publication
(10) Pub. No.: US 2001/0007940 A1
(43) Pub. Date: Jul. 12, 2001

(54) Medical device having ultrasound imaging and therapeutic means

(76) Inventors: Hosheng Tu, Newport Coast, CA (US); Olav Bergheim, Laguna Niguel, CA (US)

Correspondence Address: HOSHENG TU, 26061 MERIT CIRCLE #101, LAGUNA HILLS, CA, 92653, US

(21) Appl. No.: 09/798,568

(22) Filed: Mar. 2, 2001

Related U.S. Application Data
(63) Continuation-in-part of application No. 09/334,503, filed on Jun. 21, 1999, now Pat. No. 6,235,024.

(51) Int. Cl. A61B 18/14; A61B 8/00; A61B 8/12; A61B 8/14

(52) U.S. Cl. 606/41; 600/439

(57) ABSTRACT

An improved ultrasound device system comprising an ultrasound transducer at about a distal section of an elongate apparatus, wherein the ultrasound transducer comprises the capabilities of ultrasound imaging, RF thermal therapy, cryogenic therapy and temperature sensing for effective treating the tissue or lesion.

### RELATIONSHIP TO COPENDING APPLICATION

This application is a continuation-in-part application of patent application Ser. No. 09/334,503 filed Jun. 21, 1999, entitled “Catheter System Having Dual Ablation Capability”, now allowed, the entire contents of which are incorporated herein by reference.

### TECHNICAL FIELD OF THE INVENTION

The present invention generally relates to improved constructions for a therapeutic device system and its use thereafter. More particularly, this invention relates to an ultrasound imaging apparatus having hyperthermic and/or hypothermic therapy means for tissues treatment.

### BACKGROUND OF THE INVENTION

An artery is one of the tube-shaped blood vessels that carry blood away from a heart to the body's tissues and organs. An artery is made up of an outer fibrous layer, a smooth muscle layer, connecting tissues and inner lining cells. If arterial walls become hardened due to the accumulation of fatty substances, then blood flow can be diminished. Hardening of the arteries, or loss of vessel elasticity, is termed arteriosclerosis while fatty deposit build-up is termed atherosclerosis. Atherosclerosis and its complications are a major cause of death in the United States. Heart and brain diseases are often the direct result of this accumulation of fatty substances that impair the arteries' ability to nourish vital body organs.

Balloon angioplasty is a nonsurgical method of clearing coronary and other arteries, blocked by atherosclerotic plaque, fibrous and fatty deposits on the walls of arteries. A catheter with a balloon-like tip is threaded up from the arm or groin through an artery until it reaches the blocked area. The balloon is then inflated, flattening the plaque and increasing the diameter of the blood vessel opening. The arterial passage is thus widened. As a result of enlarging the hardened plaque, cracks may unfortunately occur within the plaque to expose the underlying fresh tissue or cells to the blood stream.

There are limitations, however, to this technique's application, depending on the extent of the disease, the blood flow through the artery, and the part of the anatomy and the particular vessels involved. Plaque build-up and/or severe restenosis recurrence within 6 months is up to 30-40 percent of those treated. Balloon angioplasty can only be characterized as a moderate-success procedure. Recently, a newer technique of inserting a metallic stenting element, e.g. a coronary stent, is used to permanently maintain the walls of the vessel treated at its extended opening state. Vascular stents are tiny mesh or coil tubes made of stainless steel or other metals and are used by heart surgeons to prop open the weak inner walls of diseased arteries. They are often used in conjunction with balloon angioplasty to prevent restenosis after the clogged arteries are treated. Stenting technique reduces the probability of restenosis; however, the success rate is still suboptimal. The underlying fresh tissue or cells after angioplasty/stenting procedures still pose as a precursor for vessel reclosures, restenosis, or angio-spasm.

One major drawback with angioplasty and/or stenting is that they open up the plaque or the obstruction and expose the underlying collagen or damaged endothelium to the blood flow. Fresh collagen has pro-thrombotic and platelet-affinity properties that are part of body's natural healing processes. Unless the collagen or the damaged endothelium is passivated or modulated, the chances for blood vessel clotting as well as restenosis always exist. Moderate focal heat is known to tighten and shrink the collagen tissue. It is also clinically verified that thermal energy (either hyperthermic or hypothermic) is capable of denaturing the tissue and modulating the collagenous molecules in such a way that treated tissue becomes more resilient. Therefore, it becomes imperative to post-treat vessels walls after the walls are treated with angioplasty, stenting, or atherectomy procedures.

Intravascular ultrasound (IVUS) is a valuable adjunct to angiography, providing new insights in the diagnosis for coronary diseases. The high frequency ultrasound probe has the capability of imaging the atherosclerotic plaque, particularly the vulnerable prone-to-rupture plaque. However, after identifying and imaging the plaque site, the conventional IVUS catheter is generally withdrawn from the vessel and a treatment catheter is inserted into the same vessel. Unfortunately, the vulnerable site is very unlikely to be identified again by the treatment catheter unless the treatment catheter also has imaging capability. The treatment means might include hyperthermic therapy, hypothermic therapy or other local drug delivery.

One type of thermal therapies is radiofrequency (RF) ablation, which requires tissue contact. Another type of thermal therapies is cryogenic ablation, which utilizes the Peltier effect on a junction of two wires with different electromotive potentials. RF therapeutic protocol has been proven to be highly effective when used by electrophysiologists for the treatment of tachycardia; by neurosurgeons for the treatment of Parkinson's disease; and by neurosurgeons and anesthetists for other RF procedures such as Gasserian ganglionectomy for trigeminal neuralgia and percutaneous cervical cordotomy for intractable pains. Radiofrequency treatment, which exposes a patient to minimal side effects and risks, is generally performed after first locating the tissue sites for treatment; that is, an ultrasound imaging system of this invention. Thermal energy, when coupled with a temperature control mechanism, can be supplied precisely to the apparatus-to-tissue contact site to obtain the desired thermal energy for treating a tissue.

Another type of tissue ablation might include “cold therapy”. Larsen et al. in U.S. Pat. No. 5,529,067 discloses methods and apparatus for use in procedures related to the electrophysiology of the heart. Specifically, Larsen et al. discloses an apparatus having thermocouple elements of different electromotive potential conductively connected at a junction, whereby an electrical current is passed through the thermocouple elements to reduce the temperature of the junction in accordance with the Peltier effect and thereby cool the contacted tissue. A detailed description of the Peltier effect and an apparatus utilizing the Peltier effect is set forth in U.S. Pat. No. 4,860,744 entitled “Thermoelectrically Controlled Heat Medical Catheter” and in U.S. Pat. No. 5,529,067 entitled “Methods For Procedures Related to the Electrophysiology of the Heart”, both of which are incorporated by reference herein.

The above-mentioned prior art has the advantage of using the device as a treatment apparatus, but they do not provide any means for identifying or imaging the lesion or the target tissue site for treatment.

Other situations may arise where it is advantageous or desirable to combine the benefits of ultrasound imaging for identifying the lesion site and a cryogenic therapy to the target tissue or a RF current therapy for providing focal thermal energy site-specifically. More particularly, it is highly desirable that the ultrasound imaging and the thermal therapy be associated with the same transducer of the device system so that the exact target tissue site is continuously imaged during the treatment stage. It would be beneficial to have a device for imaging and treating tissues in a single procedure.

Human hearing can't go beyond about 18,000 vibrations per second, or 18 kHz. Higher frequencies have a shorter wavelength and are generally used for medical imaging, such as investigating a fetus in the mother's womb. Several U.S. patents disclose ultrasonic imaging or ablation and its application. Examples are U.S. Pat. No. 5,368,557 to Nita et al., U.S. Pat. No. 5,474,530 to Passafaro et al., U.S. Pat. No. 5,606,974 to Castellano et al., U.S. Pat. No. 5,676,692 to Sanghvi et al., and U.S. Pat. No. 5,827,204 to Grandia et al. However, none of the above-identified patents discloses an ultrasound transducer having a plurality of operational capabilities of ultrasonic imaging, thermal ablation, cryogenic ablation, and temperature sensing/control.

One of the drawbacks with the above-described conventional method for using an ultrasound imaging device and a separate ablation device for tissue management is that it does not allow for immediate therapeutic treatment using “the same diagnostic device” in a site-specific manner. Since a hyperthermic energy or hypothermic energy has been demonstrated effective in treating tissues, there is an urgent need for an improved device system having at least dual capabilities of ultrasound imaging, therapeutic means for effectively treating the tissues by using radiofrequency energy or cryogenic energy, and temperature sensing/control for advanced tissue treatment.

### SUMMARY OF THE INVENTION

The present invention is a method and apparatus for providing ultrasound imaging and precisely site-specific heating (and cooling in some cases) of a small region for tissue management. Such precisely site-specific tissue treatment is provided by the hyperthermic or hypothermic energy delivered from the ultrasound imaging probe of the present invention. The thermal treatment is controlled by intermittently sensing the tissue temperature by the same ultrasound probe. The ultrasound probe may be used for tissue treatment such as tumor, cancer, prostate, vulnerable plaque, atherosclerotic plaque, hemorrhoid, inflamed tissue, polyps, and the like. It may also be used for imaging and destruction of diseased tissue in various parts of the body such as intestine, colon, urethra, uterine tube, fallopian tube, vascular vessel, breast, skin, nerve, eye, bladder, brain, or the like.

The probe utilizes a thermoelectric junction which incorporates two electrical conducting wires having distinctly different electromotive potentials from each other. The thermoelectric junction forms an outer metallic surface of a transducer of the high frequency ultrasound probe. By “ultrasound probe”, it means the ultrasound transducer portion of a medical device that is used in the imaging and/or therapeutic effects. An inner metallic surface of the transducer is conductively connected to an electrical wire to the external high frequency current source. The high frequency ultrasound probe of the present invention thus has the capability of imaging the target tissue ultrasonically, treating the tissue from the thermoelectric junction (a part of the ultrasound transducer) by hyperthermic radiofrequency or Peltier effect hypothermia, and sensing the tissue temperature by the thermoelectric junction as a thermocouple sensor. Each of the conducting wires has an electrically insulative jacket that insulates the respective wires along their lengths to prevent short circuiting.

Accordingly, it is an object of the present invention to provide a method and an improved apparatus for imaging the tissue ultrasonically and treating the vascular vessels, or other tissues, such as intestine, colon, urethra, uterine tube, bladder, skin, nerve, brain, stomach, fallopian tube, liver, and the like using thermal energy hyperthermically or hypothermically from the ultrasound transducer. It is another object of the present invention to provide an ultrasound catheter having at least dual capabilities of ultrasound imaging, thermal ablation, and temperature sensing from the same transducer material.

In one embodiment, the method for tissue management comprises imaging the tissue with a high frequency ultrasound probe and treating the tissue with radiofrequency energy or cryogenic energy associated from the high frequency ultrasound probe. The frequency of the high frequency ultrasound probe may be in the range of 1 MHz to 100 MHz. The target diseased tissue may be selected from a group consisting of tumor, cancer, prostate, vulnerable plaque, atherosclerotic plaque, hemorrhoid, and inflamed tissue. Furthermore, the high frequency ultrasound probe may be an intravascular ultrasound (IVUS) catheter. The tissue may be selected from a group consisting of intestine, colon, urethra, uterine tube, fallopian tube, vascular vessel, breast, skin, nerve, eye, bladder, and brain. The operational mode for imaging the tissue, treating the tissue, and/or sensing tissue temperature may be conducted in an alternative manner, in a sequential manner, in a preprogrammed manner, or randomly at the discretion of a user. The method further comprises sensing tissue temperature with the thermoelectric junction portion of the high frequency ultrasound probe.

The high frequency ultrasound probe of the present invention may comprise (a) an ultrasound transducer having a wall with inner and outer surfaces, wherein an inner metallic layer is formed on the inner surface and an outer metallic layer is formed on the outer surface of the transducer to conduct electrical signals to the transducer for excitation of the transducer; (b) at least one inner electrical wire for conducting electrical signal to and from the inner metallic layer; (c) a first and a second outer electrical wires for conducting electrical signal to and from the outer metallic layer, wherein the first and second outer electrical wires having different electromotive potential conductively connected at the outer metallic layer for reducing the temperature of the outer metallic layer in accordance with Peltier effect by passing an electrical current through the outer electrical wires. According to the present invention, the method further comprises conducting electrical signals to the transducer for excitation of the transducer by one of the at least one inner electrical wire and the first or second outer electrical wire from an ultrasound current source.

In a preferred embodiment, the conductivity of the first electrically conducting material for the outer metallic layer is optionally higher than the conductivity of the second electrically conducting material for the inner metallic layer. This may facilitate the radiofrequency ablation using the outer layer only because RF requires tissue contact. The inner layer and the outer layer may be made of a process selected from the group consisting of metallic coating, metallic deposition, metallic spraying, metallic imprinting, and metallic bonding. The inner layer and the outer layer may be made of a material selected from the group consisting of gold, silver, nickel, aluminum, tungsten, platinum, magnesium, and an alloy of their mixtures.

The device system of the present invention further comprises a high frequency current generator means for providing a radiofrequency current to the outer metallic layer through a first outer electrical wire and/or providing an ultrasound current to the transducer through one outer electrical wire and one inner electrical wire of the electrical conducting means. The high frequency current is provided from the high frequency current generator to the transducer in any convenient operating mode.

In one preferred embodiment of radiofrequency ablation operations, a DIP (dispersive indifferent pad) type pad or electrode, that contacts the patient, is connected to the Indifferent Electrode Connector on a RF high frequency current generator. Therefore, the RF current delivery becomes effective when a close circuit from a RF generator through an ablation element and a patient, and returning to the RF generator is formed. When using a high frequency current outlet, the generator should be grounded to avoid electrical interference. Heat is controlled by the power of the high frequency energy generator means and by the delivery duration. The standard high frequency current generator means and its applications to a patient are well known to one who is skilled in the art.

The method and apparatus of the present invention has several significant advantages over other known systems or techniques to treat a diseased tissue or lesion. In particular, the apparatus comprising an ablation element on top of an ultrasound imaging transducer results in a precisely site-specific therapeutic effect, which is highly desirable in its intended application on the atherosclerosis vulnerable plaque, tumor and other diseased tissues.

### BRIEF DESCRIPTION OF THE DRAWINGS

Additional objects and features of the present invention will become more apparent and the invention itself will be best understood from the following Detailed Description of Exemplary Embodiments, when read with reference to the accompanying drawings.

FIG. 1 is a schematic diagram of a treatment method comprising ultrasound imaging and thermal ablations in relation to a target tissue of a patient.

FIG. 2 is an overall view of a preferred ultrasound catheter system with a transducer element having imaging and ablation capabilities, constructed in accordance with the principles of the present invention.

FIG. 3 is a side cross-sectional view of the distal end portion of a preferred ultrasound catheter, having ultrasonic imaging and ablation capabilities.

FIG. 4 is a front cross-sectional view of the distal end portion of a preferred ultrasound catheter, section I-I of FIG. 3, having an ultrasound transducer with a plurality of functions.

FIG. 5 is an intravascular ultrasound catheter with a transducer according to the principles of the present invention.

FIG. 6 is a detailed description for a transducer element as shown in FIG. 5.

FIG. 7 is a simulated view of operating an intravascular ultrasound catheter of the present invention for imaging and treating a tissue inside a blood vessel.

### DETAILED DESCRIPTION OF EXEMPLARY EMBODIMENTS

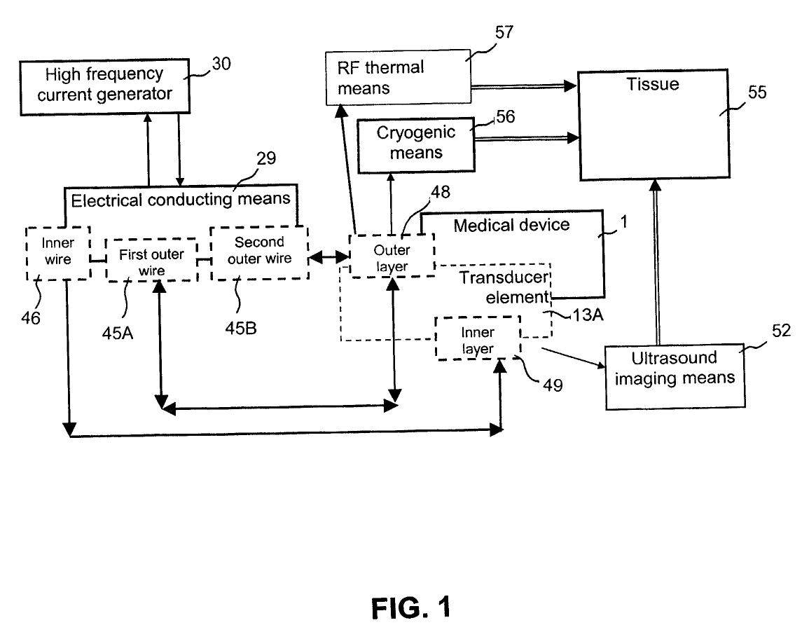

Referring to FIGS. 1 to 7, what is shown is a preferred embodiment of the present medical device system and methods, comprising imaging the tissue with a high frequency ultrasound probe and treating the tissue with hypothermic and/or hyperthermic energy derived from the same high frequency ultrasound probe.

Intravascular ultrasound (IVUS) imaging catheter uses miniaturized piezoelectric transducers and delivers ultrasound current to catheters that were approximately 1 mm in diameter. The equipment required to perform intracoronary ultrasound consists of two major components, a catheter incorporating a miniaturized transducer and a console containing the electronics necessary to reconstruct the image. High ultrasound frequencies are used, typically in the range of 1 MHz to 100 MHz. However, the high frequency ultrasound is not limited to 100 MHz. More preferably, the high frequency ultrasound is in the range of 30 to 80 MHz and provides excellent theoretical resolution. At 30 MHz, the wavelength is 50 μm.

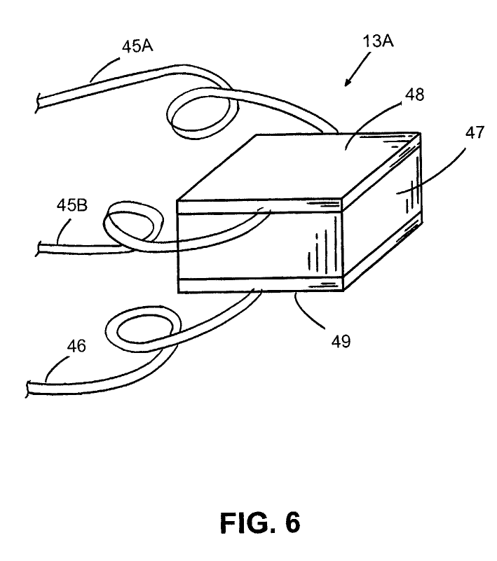

FIG. 1 shows a schematic diagram of a treatment method comprising ultrasound imaging and an ultrasound transducer element in relation to a tissue 55 of a patient. The “ultrasound transducer” as referred in the present invention usually consists of at least one transducer element, and the ultrasound transducer is typically mounted at a distal section of a medical device, such as a catheter, an apparatus, a probe, a handpiece, or the like. In a preferred embodiment, a high frequency current generator source 30 is connected to the ultrasound transducer element 13A of an apparatus or medical device 1 through an electrical conducting means 29. In one embodiment, the electrical conducting means 29 comprises a plurality of conducting wires 45A, 45B, 46. The transducer element 13A as detailed in FIG. 6 comprises a wall 47 with an outer surface and an inner surface. An outer metallic layer 48 is formed on the outer surface of the transducer element and an inner metallic layer 49 is formed on the inner surface of the transducer element. When both metallic layers are connected to an external high frequency generator source, the transducer element is excited to perform the ultrasound imaging function. The wall 47 may be made of a piezoelectric material or a thin film piezo-ceramic material.

The electrical conducting means 29 has at least one inner electrical wire 46 for conducting electrical signal to and from the inner metallic layer 49. The electrical conducting means 29 also has a first outer electrical wire 45A and a second outer electrical wire 45B for conducting electrical signal to and from the outer metallic layer 48, wherein the first and second outer electrical wires 45A, 45B have different electromotive potential conductively connected at the outer metallic layer 48 for reducing the temperature of the outer metallic layer in accordance with Peltier effect by passing an electrical current through the outer electrical wires. Typically, one of the outer electrical wires may be made of the same conducting material as that for the inner electrical wire 46.

The high frequency current generator means 30 provides, in one embodiment, high frequency current in RF ablation means 57 to the outer layer 48 of the transducer element 13A through a first outer electrical wire 45A of the electrical conducting means 29. This monopolar RF mechanism uses the patient as the returning electrode route for a complete electrical circuit. The high frequency current generator 30 can also provides, in another embodiment, an ultrasound imaging means 52 through the inner electrical wire 46 and the first outer electrical wire 45A or the second outer electrical wire 45B by a conventional ultrasound emitting and receiving mechanism. Furthermore, the method and devices of the present invention provide, in still another embodiment, cryogenic ablation means 56 for treating tissue by the Peltier effect through the first outer electrical wire 45A and the second outer electrical wire 45B. All electrical wires of the present invention are insulated or covered with an insulating material. The Peltier effect has been fully described in U.S. Pat. No. 4,860,744, the entire contents of which are incorporated herein by reference.

In an alternate embodiment, the outer metallic layer 48 is in close contact with the underlying tissue 55. As in the case of RF thermal means 57, a DIP (dispersive indifferent pad) type pad, that contacts the patient, is connected to the Indifferent Electrode Connector on the high frequency generator means 30 through a returning electrical wire. Therefore, the high frequency hyperthermic energy delivery becomes effective when a close circuit from a generator 30 through a patient and returning to the generator is formed. The outer metallic layer 48 functions as the junction of a thermocouple-type temperature sensor and is used to measure the tissue temperature. Thermal energy is controlled by the power of the energy associated and by the operational duration. In the case of hypothermic energy ablation, the temperature lowering of the outer metallic layer 48 is in accordance with Peltier effect by passing an electrical current through the two outer electrical wires, wherein the first 45A and second 45B outer electrical wires have different electromotive potential conductively connected at the outer metallic layer junction 48.

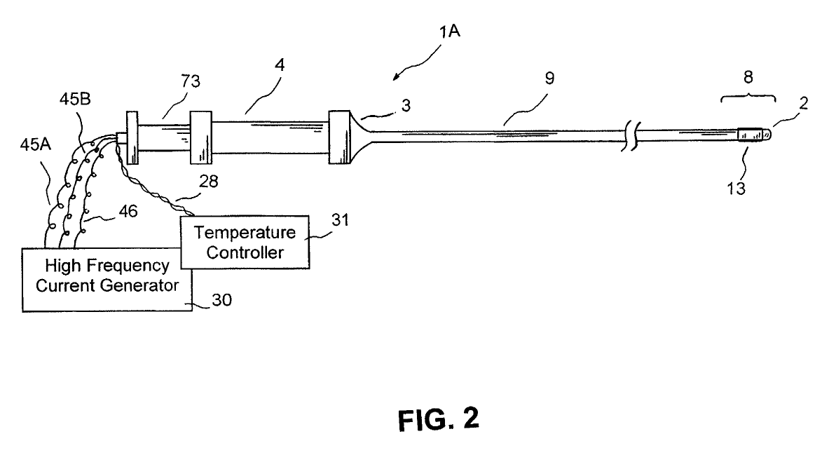

As shown in an exemplary embodiment in FIG. 2, the high frequency ultrasound catheter 1A in the form of an elongate catheter tubular assembly comprises an elongate catheter tubing 9 having a distal section 8, a distal end 2, a proximal end 3, and at least one lumen 10 extending therebetween. A handle 4 is attached to the proximal end 3 of the elongate catheter tubing 9, wherein the handle 4 has a cavity. An ultrasound transducer 13 is disposed at the distal section 8.

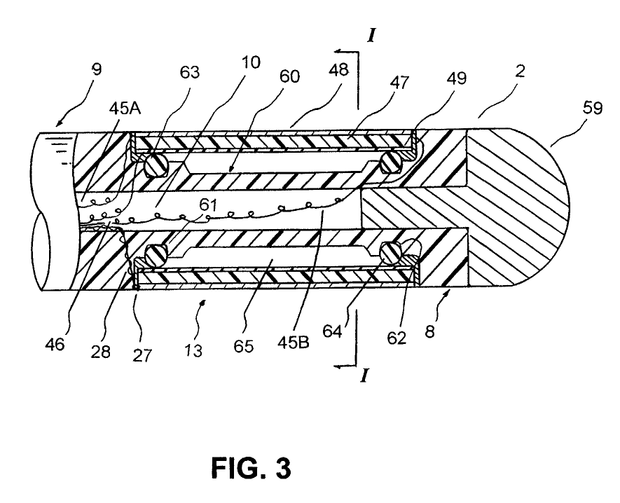

FIG. 3 shows a side cross-sectional view of the distal end portion of a high frequency ultrasound probe or catheter 1A, having an ultrasound transducer 13 with dual imaging and ablation capabilities. An additional temperature sensor 27 may be mounted at close proximity of the transducer 13 for auxiliary temperature calibration purposes. In one embodiment, the transducer 13 comprises a cylindrical transducer piezoelectric material 47 having an outer metallic layer 48 and an inner metallic layer 49.

The inner metallic layer and the outer metallic layer may be made of a manufacturing process selected from the group consisting of metallic coating, metallic deposition, metallic spraying, metallic bonding, metallic printing, and the like. The inner and the outer metallic layers may be made of a material selected from the group consisting of gold, silver, nickel, aluminum, tungsten, platinum, magnesium, and an alloy of their mixtures. In an alternate preferred embodiment, the conductivity of the outer metallic layer is different from that of the inner metallic layer.

It has been noted that piezoelectric transducers may effect a pumping action of fluid through an associated orifice or opening due to the movement of the transducer.

Fluid entry into the interior of an ultrasound catheter or probe is undesired. A cap 59 at the distal end 2 of the elongate catheter tubing 9 is to prevent liquid leakage.

In general, the transducer 13 is mounted to a mounting base 60 and the mounting to base is part of the distal section 8 of the elongate catheter tubing 9. The mounting base 60 has two annual O-ring retention grooves 61, 62. The transducer 13 is then mounted over the O-rings 63, 64 which are positioned inside the O-ring retention grooves 61 and 62. The O-rings may be made of low Durometer material such as silicone or polyurethane. The space 65 between the mounting base 60 and the transducer 13 is so designed and spaced for absorbing the vibration from the piezoelectric transducer material 47. In one particular embodiment, the space 65 is in an essentially vacuum state because ultrasound cannot transmit through a vacuum.

A high frequency current generator means 30 is part of the ultrasound probe system 1A or the ultrasound medical device 1, wherein an electrical conducting means 29 is coupled from the generator 30 to the ultrasound transducer 13. The high frequency current generator means 30 may comprise a switch means for switching high frequency energy to radiofrequency spectrum, ultrasound frequency spectrum, radiofrequency/ultrasound frequency overlapped spectrum, or spectrum for Peltier effect. This switch means is an operator-initiated action to the appropriate operating mode selected from the group consisting of radiofrequency ablation mode, ultrasound imaging mode, cryogenic ablation mode, temperature sensing mode, or combination thereof. In each mode or a combination of the modes, the energy delivery may be continuous, pulsed, programmed, randomly, and the like.

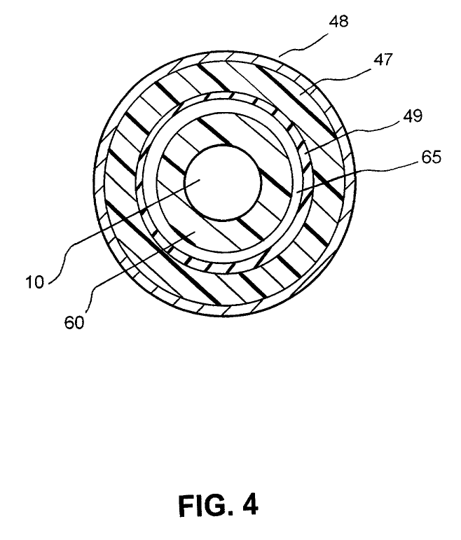

FIG. 4 shows a front cross-sectional view of the distal end portion of a preferred ultrasound catheter probe, section I-I of FIG. 3, having an ultrasound transducer element with dual ultrasonic imaging and ablation capabilities. The transducer 13 includes the outer metallic layer 48, the transducer material 47 and the inner metallic layer 49. The first insulated outer electrical wire 45A of the electrical conducting means 29 is at one end coupled to one side of the outer metallic layer 48 and at another end coupled to the high frequency generator means 30. Similarly, the second outer electrical wire 45B of the electrical conducting means is at one end coupled to an opposite side of the outer metallic layer 48 with reference to the coupling site of the first outer wire 45A.

On one hand, the electrical circuit from the generator 30 through the first outer wire 45A to the outer layer 48 constitutes a portion of the monopolar radiofrequency ablation mode, while the remaining portion of the circuit comprises a tissue 55, a PID pad, a returning wire and the generator 30. The second insulated electrical wire 46 of the electrical conducting means 29 is at one end coupled to the inner layer 49 and at another end coupled to the generator 30. The ultrasound imaging is accomplished by energizing the transducer 13 from both surfaces.

The catheter probe system of the present invention may further comprise a steering mechanism 73 at the handle 4 for controlling deflection of the distal tip section 8 of the elongate catheter tubing 9. Usually a rotating ring or a push-pull plunger is employed in the steering mechanism. In another embodiment, the steerable ultrasound catheter comprises bidirectional deflection of the distal tip section perpendicular to the catheter tubing. In an exemplary embodiment, the means for deflecting the distal portion of the catheter comprises at least one steering wire along with a flat wire. Said steering wires are attached to radially offset locations at the distal end of the deflectable portion of the catheter tubing whereas at least a flat wire radially offset the steering wires, and means at the proximal end of the tubing for selectively applying tension to the steering wires to cause deflection of the deflectable tip section. In some cases, the function of a flat wire can be substituted by a spring coil that is stationary at its proximal end with respect to the tubing. Usually the means for selectively applying tension comprises a handle, and means for applying tension to the steering wire comprises a rotatable ring or a push-pull button disposed on the handle, the ring or button being coupled to the proximal end of a steering wire. A variety of other tensions applying mechanisms, such as joysticks, may also be employed. The steering mechanism and its construction in a catheter is well known to one who is skilled in the art.

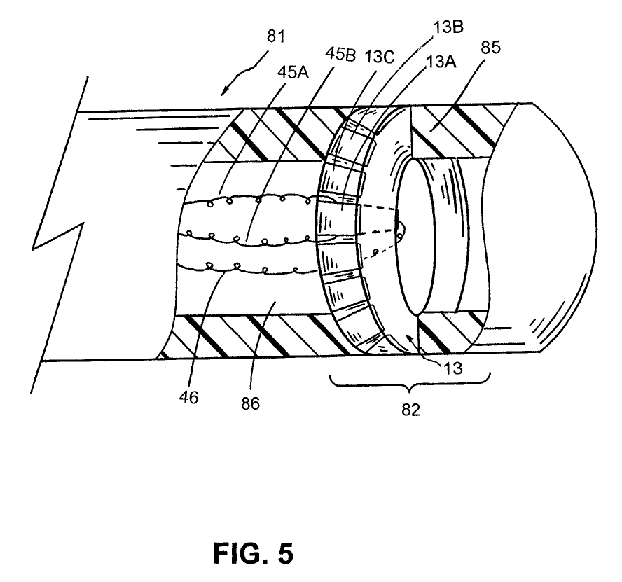

In one example of IVUS system, multiple transducer elements up to 64 in an annular array are activated sequentially to generate the image. Multiple-element designs typically result in catheters that are easier to set up and use as shown in FIG. 5. Ultrasound provides a unique method for studying the morphology of atherosclerosis in vivo. An important potential application of intracoronary ultrasound is the identification of atheromas at risk of rupture (also known as vulnerable plaque or soft plaque). Nissen and Yock (Circulation 2001;103:604-616) report that acute coronary syndromes frequently develop in territories with minimally diseased vessels rather than high-grade stenosis. The histology of unstable or vulnerable plaques usually reveals a lipid-laden atheroma with a thin fibrous cap.

The mode of an IVUS operation is described as follows. The images portrayed by an IVUS are perpendicular cross-sections of the vascular vessel along the length of the vessel as the catheter is advanced or withdrawn. The catheters can be manually advanced over a guidewire and held in place while a particular segment of the vessel is being interrogated, or the device can be attached to a mechanical pull-back sled that withdraws the catheter at a set rate, usually 0.5 or 1 mm/second.

One desirable aspect of tissue characterization with IVUS would be the capacity to diagnose an unstable or vulnerable plaque (Tobis J, chapter 1 in a book “Techniques in Coronary Artery Stenting” published by Martin Dunitz 2000). An unstable plaque has been described as a plaque with a large lipid component covered by a thin fibrous cap. It has been hypothesized that a plaque progression is spontaneous rupture of the fibrous capsule and thrombus formation as blood mixes with the thrombogenic lipid core. Although plaque rupture occurs only sporadically, this phenomenon has been documented in vivo. It is one object of the present invention to treat the vulnerable plaque using the same IVUS catheter transducer once the plaque is diagnosed by a high frequency IVUS.

FIG. 5 shows an ultrasound transducer catheter 81 of the present invention, wherein the ultrasound transducer 13 may be mounted at a catheter tip section 82 adjacent the catheter shaft 85 to be used as an IVUS catheter. In a specific embodiment, the ultrasound transducer 13 has a plurality of transducer elements 13A, 13B, 13C etc. located around the circumference of the catheter tip section 82. The individual transducer elements are stimulated and the returning echos are integrated by miniaturized computer chips within the distal end of the catheter. The ultrasound information is then stored by an external computer for analysis. Each transducer element has at least one inner electrical wire 46 connected to the inner metallic layer 49 and at least two outer electrical wires 45A, 45B connected to the outer metallic layer 48 of a transducer element. All insulated electrical wires pass through the lumen 86 of the catheter 81 to an external high frequency current source.

As shown in FIG. 6, the ultrasound transducer element 13A may comprise a wall 47 with inner and outer surfaces, wherein an inner metallic layer 49 is formed on the inner surface and an outer metallic layer 48 is formed on the outer surface of the transducer to conduct electrical signals to the transducer for excitation of the transducer. At least one inner electrical wire 46 for conducting electrical signal to and from the inner metallic layer 49 is coupled to an external high frequency current source. There are a first outer electrical wire 45A and a second outer electrical wire 45B for conducting electrical signal to and from the outer metallic layer 48, wherein the first and second outer electrical wires have different electromotive potential conductively connected at the outer metallic layer for reducing the temperature of the outer metallic layer in accordance with Peltier effect by passing an electrical current through the outer electrical wires 45A, 45B.

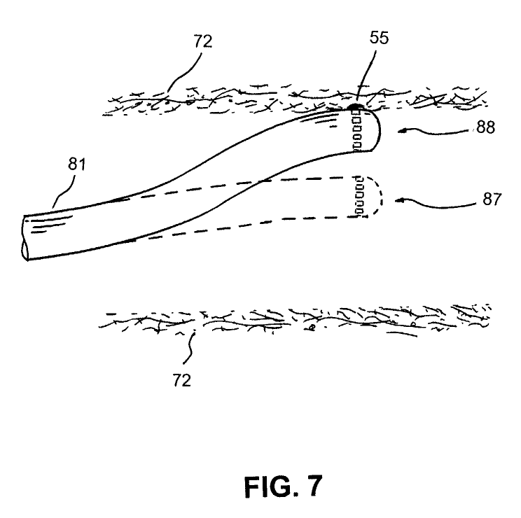

FIG. 7 shows a simulated view of placing an intravascular ultrasound catheter 81 of the present invention inside a blood vessel 72. For illustration purposes, after a vulnerable plaque or target tissue 55 is imaged or identified by the IVUS catheter 81 at the catheter position 87, the catheter may be deflected to place the outer metallic layer 48 of the transducer 13 of the catheter 81 against the target tissue at a new catheter position 88. Depending on a physician's decision, RF thermal therapy and/or cryogenic therapy can be applied to the target tissue through the outer metallic layer 48 of the transducer element 13A of the present invention. In this case, only one of the transducer elements is cryogenically energized for therapeutic ablation on the target tissue. It is also within the scope of the present invention to provide RF hyperthermic energy or cryogenic hypothermic energy to one or more transducer elements. Intermittently, the outer metallic layer junction can be used as a thermocouple to sense the tissue temperature for feedback controlling the thermal therapy.

### MODE OF OPERATION EXAMPLES

As shown in FIGS. 5, 6 and 7, a high frequency IVUS catheter may be inserted into a coronary artery through an opening at the femoral artery or vein. Following the standard ultrasound imaging techniques as taught by Tobis (chapter 1 in the book “Techniques in Coronary Artery Stenting” published by Martin Dunitz 2000), the intravascular ultrasound image is taken. To obtain 3-D ultrasound data along the blood vessel, the IVUS catheter could be attached to a mechanical pull-back sled that withdraws the catheter at a set rate, usually 0.5 or 1.0 mm per second. The catheter can also optionally track over a central core wire for insertion and withdrawal. There are several possible operational modes as outlined below:

1. Image and identify the lesion site using the standard ultrasound imaging technique of the present invention with high ultrasound frequency;

2. Steer the ultrasound transducer to approach the lesion site by continuously imaging the tissue;

3. Ensure that the outer metallic layer of the transducer contacts the lesion site by ultrasound imaging technique as described;

4. Apply RF hyperthermic therapy through the outer metallic layer to the lesion while continuously monitor the tissue temperature for feedback control; and/or

5. Apply cryogenic hypothermic therapy through the outer metallic layer to the lesion site while continuously monitor the tissue temperature for feedback control.

6. Optionally, perform a combination of the operational modes as listed above.

From the foregoing description, it should now be appreciated that an ultrasound transducer device system and methods for imaging and treating tissues have been disclosed. While the invention has been described with reference to a specific embodiment, the description is illustrative of the invention and is not to be construed as limiting the invention. Various modifications and applications to treating the tissue selected from the group consisting of intestine, colon, urethra, uterine tube, fallopian tube, vascular vessel, breast, skin, nerve, eye, bladder, liver, and brain may occur to those who are skilled in the art, without departing from the true spirit and scope of the invention, as described by the appended claims.

What is claimed is:

1. A method for tissue treatment, the method comprising imaging the tissue with a high frequency ultrasound probe and treating the tissue with radiofrequency energy delivered from said high frequency ultrasound probe.

2. The method according to claim 1, wherein frequency of the high frequency ultrasound probe is in a range of 1 MHz to 100 MHz.

3. The method according to claim 1, wherein lesion of the tissue is selected from a group consisting of tumor, cancer, prostate, vulnerable plaque, atherosclerotic plaque, hemorrhoid, polyps, and inflamed tissue.

4. The method according to claim 2, wherein the high frequency ultrasound probe is an intravascular ultrasound (IVUS) catheter.

5. The method according to claim 1, wherein the tissue is selected from a group consisting of intestine, colon, urethra, uterine tube, fallopian tube, vascular vessel, breast, skin, nerve, eye, bladder, liver, and brain.

6. The method according to claim 1, wherein imaging the tissue and treating the tissue are conducted in an alternative manner.

7. The method according to claim 1, wherein the method further comprises sensing tissue temperature with the high frequency ultrasound probe.

8. The method according to claim 7, wherein imaging the tissue, treating the tissue and sensing tissue temperature are conducted in a preprogrammed manner.

9. A method for tissue treatment, the method comprising imaging the tissue with a high frequency ultrasound probe and treating the tissue with hypothermic energy derived from said high frequency ultrasound probe.

10. The method according to claim 9, wherein frequency of the high frequency ultrasound probe is in the range of 1 MHz to 100 MHz.

11. The method according to claim 9, wherein lesion of the tissue is selected from a group consisting of tumor, cancer, prostate, vulnerable plaque, atherosclerotic plaque, hemorrhoid, polyps, and inflamed tissue.

12. The method according to claim 10, wherein the high frequency ultrasound probe is an intravascular ultrasound (IVUS) catheter.

13. The method according to claim 9, wherein the tissue is selected from a group consisting of intestine, colon, urethra, uterine tube, fallopian tube, vascular vessel, breast, skin, nerve, eye, bladder, liver, and brain.

14. The method according to claim 9, wherein the method further comprises sensing tissue temperature with the high frequency ultrasound probe.

15. The method according to claim 9, wherein the high frequency ultrasound probe comprises:
an ultrasound transducer having a wall with inner and outer surfaces, wherein an inner metallic layer is formed on the inner surface and an outer metallic layer is formed on the outer surface of the transducer to conduct electrical signals to the transducer for excitation of the transducer;
at least one inner electrical wire for conducting electrical signal to and from the inner metallic layer;
a first and a second outer electrical wires for conducting electrical signal to and from the outer metallic layer, wherein said first and second outer electrical wires have different electromotive potential conductively connected at said outer metallic layer for reducing the temperature of the outer metallic layer in accordance with Peltier effect by passing an electrical current through said outer electrical wires.

16. The method according to claim 15, the method further comprising conducting electrical signals to the transducer for excitation of the transducer by one of the at least one inner electrical wire and the first outer electrical wire from an ultrasound current source.

17. The method according to claim 15, the method further comprising sensing temperature by an external temperature sensor through the first and the second outer electric wires.

18. An ultrasound transducer comprising:
a wall with inner and outer surfaces, wherein an inner metallic layer is formed on the inner surface and an outer metallic layer is formed on the outer surface of the transducer to conduct electrical signals to the transducer for excitation of the transducer;
at least one inner electrical wire for conducting electrical signal to and from the inner metallic layer;
a first and a second outer electrical wires for conducting electrical signal to and from the outer metallic layer, wherein said first and second outer electrical wires have different electromotive potential conductively connected at said outer metallic layer for reducing the temperature of the outer metallic layer in accordance with Peltier effect by passing an electrical current through said outer electrical wires.

19. The ultrasound transducer according to claim 18, wherein electrical signals are conducted to the transducer for excitation of the transducer by one of the at least one inner electrical wire and the first outer electrical wire from an ultrasound current source.

20. The ultrasound transducer according to claim 18, wherein temperature signals of the outer metallic layer are transmitted to an external temperature sensor through the first and the second outer electrical wires for temperature sensing.
